(19) United States
(12) Patent Application Publication
(10) Pub. No.: US 2006/0024261 A1
(43) Pub. Date: Feb. 2, 2006

(54) Cosmetic compositions comprising a linear sulfonic polyester and a polyurethane, processes using these compositions and uses thereof

(76) Inventors: Cécile Bebot, Clichy (FR); Sabine Vrignaud, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/177,350

(22) Filed: Jul. 11, 2005

Related U.S. Application Data
(60) Provisional application No. 60/620,669, filed on Oct. 22, 2004.

(30) Foreign Application Priority Data
Jul. 29, 2004 (FR) 04 08397

(51) Int. Cl. A61K 8/87; A61K 8/85

(52) U.S. Cl. 424/70.11; 424/70.17

(57) ABSTRACT

The disclosure provides cosmetic compositions comprising, in a cosmetically acceptable medium, at least one water-dispersible linear sulfonic polyester and at least one thickening polyurethane in a particular ratio, processes using the cosmetic compositions, and uses of the cosmetic compositions for fixing hairstyles.

This application claims benefit of U.S. Provisional Patent Application No. 60/620,669 filed Oct. 22, 2004, the contents of which are incorporated herein by reference. This application also claims benefit of priority under 35 U.S.C. § 119 to French Patent Application No. 04 08397, filed Jul. 29, 2004, the contents of which are also incorporated by reference.

The present disclosure relates to cosmetic compositions comprising, in a cosmetically acceptable medium, at least one water-dispersible linear sulfonic polyester and at least one thickening polyurethane, and processes for using these cosmetic compositions for fixing hairstyles.

Cosmetic compositions for shaping and/or holding hairstyles may be spray compositions comprising a solution, which may be alcoholic, and of one or more components, known as fixing components, which may be polymer resins, which form connections or welds between the hairs or to coat the hairs. These fixing components are often formulated as mixtures with various cosmetic adjuvants.

These cosmetic compositions may be packaged, for example, in pump-dispenser bottles or in suitable aerosol containers pressurized using a propellant. The aerosol system may comprise a liquid phase (or fluid) and a propellant.

Styling compositions in the form of gels, creams or mousses may also be used.

Once applied to the hair, the fixing compounds should allow fixing of the hair.

However, the polymers commonly used as fixing agents in styling compositions do not allow the shape of the hairstyle to be maintained when the hair comes into contact with water for a prolonged period, for example, when the hair is contacted with rain, perspiration, or during bathing such as bathing in the sea, swimming pool, etc.

The present inventors have discovered, surprisingly and advantageously, that the use of cosmetic compositions comprising, in a cosmetically acceptable medium, at least one water-dispersible linear sulfonic polyester and at least one particular thickening polyurethane, according to one embodiment, in particular ratios of the at least one linear sulfonic polyester to the at least one polyurethane, makes it possible to fix and shape hairstyles and also to maintain the shape of the hairstyles when the hair comes into contact with water for a prolonged period.

As used herein, this phenomenon is referred to as “water resistance.”

As used herein, the term “prolonged period” means contact with water for a minimum period of one minute, for example, 10 minutes or even 20 minutes.

The compositions described herein also make it possible to obtain styling that is resistant to atmospheric moisture.

The compositions described herein allow good fixing and good hold of the hair, e.g., a styling effect that lasts throughout the day, or even for several days, which shows good water resistance, such as good resistance to repeated bathing. These compositions also have the advantage of being removable by shampoo.

The compositions also allow good cosmetic properties to be imparted to the hair.

The present disclosure provides cosmetic compositions comprising, in a cosmetically acceptable medium, at least one water-dispersible linear sulfonic polyester and at least one thickening polyurethane, in a weight ratio of the at least one linear sulfonic polyester to the at least one polyurethane ratio greater than or equal to 2.

The present disclosure also provides processes for shaping or holding hairstyles in which the cosmetic compositions described above are used.

In addition, the present disclosure provides a method of using the cosmetic compositions comprising, in a cosmetically acceptable medium, at least one water-dispersible linear sulfonic polyester and at least one thickening polyurethane in a weight ratio of the at least one linear sulfonic polyester to the at least one polyurethane ratio greater than or equal to 2, as styling compositions for fixing and/or holding the hair, for example, for when the hair comes into contact with water for a prolonged period, e.g., in the case of rain, perspiration, and during bathing such as bathing in the sea or in a swimming pool.

The methods for using the compositions allow a water-resistant hairstyle (shaped hair) to be obtained.

The cosmetic compositions may be in any art recognized form, e.g., lotions, sprays, mousses, gels, creams, etc.

Other subjects, characteristics, aspects and advantages will emerge even more clearly on reading the description and the example that follow.

As used herein, the term “styling cosmetic composition” means a composition for shaping and/or holding a hairstyle.

As used herein, the term “thickening polyurethane” means a polyurethane which, in solution or in dispersion in water at a concentration of 2% and at 25° C., gives the aqueous solution or the dispersion a viscosity of greater than 250 cp, measured using a Rheomat 180 type viscometer.

The cosmetically acceptable medium used in the compositions may be an aqueous or aqueous-alcoholic medium optionally comprising at least one additional organic solvent.

Alcohols that may be used in the compositions include monohydroxylated alkanols chosen from C₁-C₄ lower alcohols, such as ethanol, isopropanol, tert-butanol, and n-butanol, for example, ethanol.

The alcohol concentration in the compositions ranges from 0 to 20%, for example, from 0 to 10%, such as from 0 to 5% by weight relative to the total weight of the composition.

Optionally, the compositions contain no C₁-C₄ alcohol.

Additional organic solvents that may be used in the compositions include, but are not limited to, polyols, for example, propylene glycol, polyol ethers, and mixtures thereof.

The concentration of additional organic solvent in the compositions may range from 0 to 30%, for example from 0 to 20%, by weight relative to the total weight of the composition.

Water-Dispersible Linear Sulfonic Polyesters

The compositions comprise a water-dispersible linear sulfonic polyester.

As used herein, the term “water-dispersible linear sulfonic polyester” means any sulfonic polyester capable of forming a dispersion, i.e., a two-phase system in which the first phase is formed from finely divided particles uniformly distributed in the second phase continuous phase.

As used herein, the term “sulfonic polyester” means copolyesters obtained by polycondensation of at least one dicarboxylic acid or an ester thereof, of at least one diol, and of at least one sulfoaryldicarboxylic difunctional compound substituted on the aromatic nucleus with an —SO₃M group wherein M is chosen from a hydrogen atom or a metal ion such as Na⁺, Li⁺, and K⁺.

The water-dispersible linear sulfonic polyesters may have a weight-average molecular mass ranging from about 1000 to 60,000, for example, ranging from 4000 to 20,000, as determined by gel permeation chromatography (GPC).

The glass transition temperature (Tg) of the sulfonic polyesters may range from 10 to 100° C. For example, the Tg of the polyester or polyesters used may be greater than or equal to 50° C.

The Tg is measured by differential thermal analysis (DSC, differential scanning calorimetry) according to ASTM standard D3418-97.

Sulfonic polyesters are described in greater detail in U.S. Pat. Nos. 3,734,874; 3,779,993; 4,119,680; 4,300,580; 4,973,656; 5,660,816; 5,662,893; and 5,674,479.

The sulfonic polyesters may comprise one or more units derived from isophthalic acid, sulfoaryidicarboxylic acid salt and diethylene glycol, for example, the sulfonic polyesters may be obtained from isophthalic acid, the sodium salt of sulfoisophthalic acid, diethylene glycol, and 1,4-cyclohexanedimethanol.

Examples of sulfonic polyesters also include those known under the INCI name diglycol/CHDM/isophthalates/SIP, and sold under the trade name Eastman AQ Polymer (AQ35S, AQ38S, AQ55S, and AQ48 Ultra) by Eastman Chemical.

The concentration of water-dispersible linear sulfonic polyesters used in the compositions may range from 0.1 to 40%, for example, from 1 to 30% or from 5 to 25%, by weight relative to the total weight of the composition.

Thickening Polyurethanes

The compositions comprise at least one thickening polyurethane.

Thickening polyurethanes that may used include anionic, cationic, amphoteric, and nonionic polyurethanes.

The thickening polyurethanes may comprise at least one fatty chain comprising at least 8 carbon atoms.

Such thickening polyurethanes show associative polymer behaviors.

Cationic Thickening Polyurethanes

Cationic thickening polyurethanes that may be used include:
cationic associative polyurethanes, described in French Patent Application No. 00/09609 and represented by the formula (I) below:
   R—X—(P)ₙ-[L-(Y)ₘ]ᵣ-L′-(P′)ₚ—X′—R′ (I)
wherein:
R and R′, which may be identical or different, each are chosen from a hydrophobic group and a hydrogen atom;
X and X′, which may be identical or different, each are chosen from a group comprising an amine function optionally bearing a hydrophobic group, and alternatively a group L″;
L, L′ and L″, which may be identical or different, each are chosen from a group derived from a diisocyanate;
P and P′, which may be identical or different, each are chosen from a group comprising an amine function optionally bearing a hydrophobic group;
Y is a hydrophilic group;
r is an integer ranging from 1 to 100, for example, ranging from 1 to 50 or from 1 to 25;
n, m and p each range, independently of each other, from 0 to 1000;
and wherein the molecule comprises at least one protonated or quaternized amine function and at least one hydrophobic group.

In some embodiments, the only hydrophobic groups of these polyurethanes are the R and R′ groups at the chain ends.

In some embodiments, the cationic associative polyurethanes used correspond to formula (I) described above wherein:
R and R′ each independently are chosen from a hydrophobic group,
X and X′ each are chosen from a group L″,
n and p each range from 1 to 1000, and
L, L′, L″, P, P′, Y, m and r each have the meaning given above.

In other embodiments, the cationic associative polyurethanes used correspond to formula (I) described above wherein:
R and R′ each independently are chosen from a hydrophobic group,
X and X′ each are chosen from a group L″,
n and p are each zero, and
L, L′, L″, P, P′, Y, m and r have the meaning given above.

When n and p are 0, the polymers do not comprise units derived from a monomer comprising an amine function incorporated into the polymer during the polycondensation. The protonated amine functions of these polyurethanes result from the hydrolysis of excess isocyanate functions, at the chain end, followed by alkylation of the primary amine functions formed with alkylating agents comprising a hydrophobic group, i.e., compounds of the type RQ or R′Q, wherein R and R′ are as defined above and Q is a leaving group such as a halide, a sulfate, etc.

In yet other embodiments, the cationic associative polyurethanes used correspond to formula (I) described above wherein:
R and R′ are each independently chosen from a hydrophobic group,
X and X′ each independently are chosen from a group comprising a quaternary amine,
n and p are each zero, and
L, L′, P, P′, Y, m and r have the meaning given above.

The number-average molecular mass of the cationic associative polyurethanes may range from 400 to 500,000, for example, from 1000 to 350,000 and from 1000 to 300,000.

As used herein, the term “hydrophobic group” means a radical or polymer comprising a saturated or unsaturated, linear or branched hydrocarbon-based chain, which may comprise one or more heteroatoms such as P, O, N, and S, or a radical comprising a perfluoro or silicone chain. When the hydrophobic group is a hydrocarbon-based radical, it may comprise at least 10 carbon atoms, for example, from 10 to 30 carbon atoms, from 12 to 30 carbon atoms, or even from 18 to 30 carbon atoms.

In some embodiments, the hydrocarbon-based group is derived from a monofunctional compound.

In some embodiments, the hydrophobic group is derived from a fatty alcohol such as stearyl alcohol, dodecyl alcohol, and decyl alcohol. It may also be a hydrocarbon-based polymer such as polybutadiene.

When X and/or X′ denote a group comprising a tertiary or quaternary amine, X and/or X′ may be chosen from one of the following formulae: 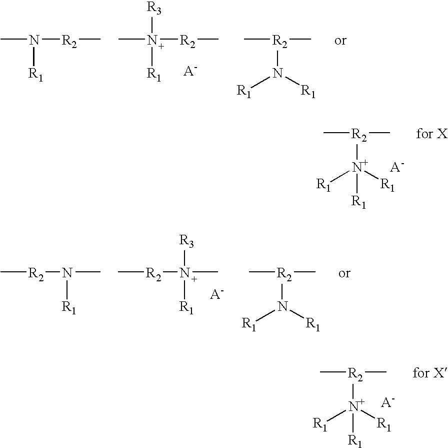
wherein:
R₂ is chosen from a linear and branched alkylene radicals having from 1 to 20 carbon atoms, optionally comprising a saturated or unsaturated ring, and arylene radicals, one or more of the carbon atoms optionally being replaced with a heteroatom chosen from N, S, O, and P;
R₁ and R₃, which may be identical or different, are each chosen from linear and branched C₁-C₃₀ alkyl and alkenyl radicals and aryl radicals, at least one of the carbon atoms optionally being replaced with a heteroatom chosen from N, S, O, and P; and
A⁻ is a physiologically acceptable counterion.

In certain embodiments, the groups L, L′ and L″ represent a group of formula: 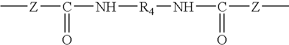
wherein:
Z, which may be identical or different, is chosen from —O—, —S— or —NH—; and
R₄ is chosen from linear and branched alkylene radicals having from 1 to 20 carbon atoms, optionally comprising a saturated or unsaturated ring, and arylene radicals, one or more of the carbon atoms optionally being replaced with a heteroatom chosen from N, S, O and P.

In some embodiments, the groups P and P′ comprising an amine function are each chosen from at least one of the following formulae: 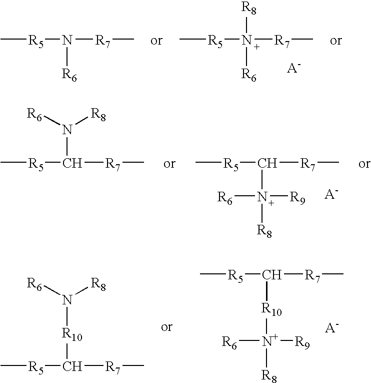
wherein:
R₅ and R₇ have the same meanings as R₂ defined above;
R₆, R₈ and R₉ have the same meanings as R₁ and R₃ defined above;
R₁₀ is chosen from linear and branched, optionally unsaturated alkylene group optionally comprising one or more heteroatom chosen from N, O, S, and P; and
A⁻ is a physiologically acceptable counterion.

As used herein with respect to Y, the term “hydrophilic group” means a polymeric or non-polymeric water-soluble group.

In certain embodiments, when Y is not a polymer, it may be chosen from ethylene glycol, diethylene glycol, and propylene glycol.

In other embodiments, when it Y is a hydrophilic polymer, it may be chosen from, for example, polyethers, sulfonated polyesters, sulfonated polyamides, and mixtures of these polymers. In certain embodiments, Y is a polyether, for example, poly(ethylene oxide) and poly(propylene oxide).

The cationic associative polyurethanes of formula (I) that may be used are formed from diisocyanates and from various compounds with functions comprising a labile hydrogen. The functions comprising a labile hydrogen may be alcohol, primary or secondary amine or thiol functions, giving, after reaction with the diisocyanate functions, polyurethanes, polyureas and polythioureas, respectively. As used herein, the term “polyurethanes” encompasses these various types of polymers, i.e., polyurethanes, polyureas, polythioureas, and copolymers thereof.

The first type of compound involved in the preparation of the polyurethanes of formula (I) is a compound comprising at least one unit comprising an amine function. This compound may be multifunctional, for example, difunctional, i.e., the compound comprises two labile hydrogen atoms borne, for example, by a hydroxyl, primary amine, secondary amine or thiol function. According to one embodiment, a mixture of multifunctional and difunctional compounds in which the percentage of multifunctional compounds is low may also be used.

As mentioned above, the compound may comprise more than one unit comprising an amine function. In this case, it is a polymer bearing a repetition of the unit comprising an amine function.

Compounds of this type may be represented by one of the following formulae:
HZ-(P)ₙ-ZH
and
HZ-(P′)ₚ-ZH
wherein Z, P, P′, n and p are as defined above.

Examples of compounds comprising an amine function that may be used include N-methyldiethanolamine, N-tert-butyldiethanolamine, and N-sulfoethyldiethanolamine.

The second type of compound involved in the preparation of the polyurethane of formula (I) is a diisocyanate having the formula:
O═C═N—R₄—N═C═O
wherein R₄ is as defined above.

Examples of diisocyanate compounds include methylenediphenyl diisocyanate, methylenecyclohexane diisocyanate, isophorone diisocyanate, toluene diisocyanate, naphthalene diisocyanate, butane diisocyanate, and hexane diisocyanate.

A third compound involved in the preparation of the polyurethane of formula (I) is a hydrophobic compound intended to form the terminal hydrophobic groups of the polymer of formula (I).

The hydrophobic compound comprises a hydrophobic group and a function comprising labile hydrogen, for example, hydroxyl, primary or secondary amines, and thiol functions.

Examples of these types of compounds include fatty alcohols such as stearyl alcohol, dodecyl alcohol, and decyl alcohol. When the compound comprises a polymeric chain, it may be, for example, α-hydroxylated hydrogenated polybutadiene.

The hydrophobic group of the polyurethane of formula (I) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, wherein R and R′ are as defined above and Q is a leaving group such as a halide, sulfate, etc.

The cationic associative polyurethane may also comprise a hydrophilic block. This block is provided by a fourth type of compound involved in the preparation of the polymer. This compound may be multifunctional, such as difunctional. According to one embodiment, it is possible to have a mixture in which the percentage of multifunctional compound is low.

The functions comprising a labile hydrogen include alcohol, primary or secondary amine, and thiol functions. These compounds may be a polymer terminated at the chain ends with one of these functions comprising a labile hydrogen.

When this compound is not a polymer, it may be made from, for example, ethylene glycol, diethylene glycol, and propylene glycol.

When the compound is a hydrophilic polymer, it may be, for example, polyethers, sulfonated polyesters, sulfonated polyamides, and mixtures of these polymers. In some embodiments, the hydrophilic compound is a polyether such as poly(ethylene oxide) and poly(propylene oxide).

According to one embodiment, the hydrophilic group termed Y in formula (I) is optional. The units comprising a quaternary amine or protonated function may suffice to provide the solubility or water-dispersibility required for this type of polymer in an aqueous solution.

In some embodiments, cationic associative polyurethanes comprising a Y group are used.

Amphoteric Thickening Polyurethanes

Amphoteric thickening polyurethanes that may be used herein include polyether polyurethanes comprising both hydrophilic blocks (such as polyoxyethylene blocks), and hydrophobic blocks (such as aliphatic sequences alone and/or cycloaliphatic and/or aromatic sequences in the chain).

The polyurethane polyethers may comprise at least two hydrocarbon-based lipophilic chains comprising from 6 to 30 carbon atoms, separated by a hydrophilic block, the hydrocarbon-based chains optionally being pendent chains, or chains at the end of the hydrophilic block. It is possible for one or more pendent chains to be included. In addition, the polymer may comprise a hydrocarbon-based chain at one end or at both ends of the hydrophilic block.

The polyurethane polyethers may be multiblock, for example, triblock. Hydrophobic blocks may be at each end of the chain, for example, a triblock copolymer with a hydrophilic central block, or distributed both at the ends and in the chain, for example, a multiblock copolymer. These polymers may also be graft polymers or starburst polymers.

Nonionic Thickening Polyurethanes

The nonionic thickening fatty-chain polyurethane polyethers may be triblock copolymers wherein the hydrophilic block is a polyoxyethylenated chain comprising from 50 to 1000 oxyethylene groups. The nonionic polyurethane polyethers comprise a urethane linkage between the hydrophilic blocks.

Nonionic fatty-chain polyurethane polyethers also include polymers in which the hydrophilic blocks are linked to the lipophilic blocks via other chemical bonds.

Examples of nonionic fatty-chain polyurethane polyethers that may be used include Rheolate 205® comprising a urea function, sold by the company Rheox, Rheolate® 208, 204 and 212, and Acrysol RM 184®.

The product Elfacos T210® comprising a C₁₂-14 alkyl chain, and the product Elfacos T212® comprising a C₁₈ alkyl chain, from Akzo may also be used.

The product DW 1206B® from Rohm & Haas comprising a C₂₀ alkyl chain and a urethane linkage, sold at a solids content of 20% in water, may also be used.

It is also possible to use solutions or dispersions of these polymers, for example, in water or in an aqueous-alcoholic medium. Examples of such polymers include Rheolate® 255, Rheolate® 278 and Rheolate® 244 sold by the company Rheox. The products DW 1206F and DW 1206J sold by the company Rohm & Haas may also be used.

Polyurethane polyethers that may be used also include those described in the article by G. Formum, J. Bakke and Fk. Hansen—Colloid Polym. Sci. 271, 380-389 (1993).

In some embodiments, a polyurethane polyether obtained by polycondensation of at least three compounds including (i) at least one polyethylene glycol comprising from 150 to 180 mol of ethylene oxide, (ii) stearyl alcohol or decyl alcohol, and (iii) at least one diisocyanate, is used.

Such polyurethane polyethers are sold by the company Rohm & Haas under the names Aculyn 44® and Aculyn 46®. Aculyn 46® is a polycondensate of polyethylene glycol comprising 150 or 180 mol of ethylene oxide, of stearyl alcohol and of methylenebis(4-cyclohexyl isocyanate) (SMDI), at 15% by weight in a matrix of maltodextrin (4%) and water (81%). Aculyn 44® is a polycondensate of polyethylene glycol comprising 150 or 180 mol of ethylene oxide, of decyl alcohol and of methylenebis(4-cyclohexylisocyanate) (SMDI), at 35% by weight in a mixture of propylene glycol (39%) and water (26%).

The thickening polyurethane polymers of the invention may be nonionic.

According to one embodiment, it is possible to obtain compositions comprising a water-dispersible linear sulfonic polyester and a nonionic thickening polyurethane that are transparent or translucent as well.

As used herein, the term “transparent or translucent compositions” means compositions having a turbidity, measured according to the method described below, of less than 800 NTU (nephelometric turbidity units), for example, less than 500 NTU.

The turbidity may be measured using a 2100 P model turbidimeter from the company Hach™ at room temperature (about 25° C.). The tubes used for the measurement are referenced AR 397A cat 24347-06. The machine is calibrated with formazine suspensions of different concentrations.

The concentration of the at least one thickening polyurethane used in the compositions according to the present invention may range from 0.01 to 10%, for example, 0.05 to 5%, such as from 0.1 to 2%, by weight relative to the total weight of the composition.

The water-dispersible linear sulfonic polyester/thickening polyurethane ratio may range from 2 to 50%, for example, from 2.5 to 20%, such as from 3% to 15%.

Additional Cosmetic Adjuvants

The compositions described herein may also comprise one or more additional cosmetic adjuvants such as those mentioned below.

Additional Fixing Polymers

Additional anionic, cationic, amphoteric, and nonionic fixing polymers and mixtures thereof known in the art may be used in the compositions described herein.

The fixing polymers may be soluble or insoluble in the cosmetically acceptable medium and may be used in the form of dispersions of solid or liquid polymer particles (e.g., lattices or pseudolattices).

Anionic Fixing Polymers

The anionic fixing polymers generally used are polymers comprising groups derived from carboxylic acid, sulfonic acid or phosphoric acid and have a number-average molecular mass ranging from 500 to 5,000,000.

The carboxylic groups may be provided by unsaturated monocarboxylic or dicarboxylic acid monomers such as those corresponding to the formula: 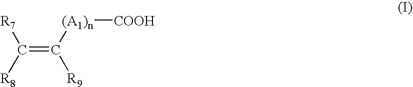
wherein:
n is an integer ranging from 0 to 10;
A₁ is a methylene group, optionally connected to the carbon atom of the unsaturated group, or to the neighboring methylene group when n is greater than 1, via a heteroatom such as oxygen or sulfur,
R₇ is chosen from a hydrogen atom, a phenyl group, or benzyl group,
R₈ is chosen from a hydrogen atom or a lower alkyl or carboxyl group, and
R₉ is chosen from a hydrogen atom and lower alkyl, —CH₂COOH, phenyl and benzyl groups.

In some embodiments, the lower alkyl group is a group having from 1 to 4 carbon atoms, such as a methyl or ethyl group.

The anionic fixing polymers may be chosen from homopolymers and copolymers of acrylic and methacrylic acid and salts thereof, crotonic acid copolymers, copolymers of C₄-C₈ monounsaturated carboxylic acids and anhydrides, polyacrylamides comprising carboxylate groups, homopolymers or copolymers comprising sulfonic groups, anionic polyurethanes, and anionic grafted silicone polymers.

The anionic fixing polymers comprising carboxylic groups include:

(A) acrylic and methacrylic acid homopolymers and copolymers, and salts thereof, such as the products sold under the names Versicol® E and K by the company Allied Colloid and Ultrahold® by the company BASF, copolymers of acrylic acid and of acrylamide sold in sodium salt form under the names Reten 421, 423 and 425 by the company Hercules, and sodium salts of polyhydroxycarboxylic acids.

(B) copolymers of acrylic or methacrylic acid with a monoethylenic monomer such as ethylene, styrene, vinyl esters, acrylic, and methacrylic acid esters, optionally grafted onto a polyalkylene glycol such as polyethylene glycol and optionally crosslinked. Such polymers are described in French Patent No. 1,222,944 and German Patent Application No. 2,330,956, the copolymers of this type comprising an optionally N-alkylated and/or hydroxyalkylated acrylamide unit in their chain as described in Luxembourg Patent Application Nos. 75370 and 75371, and are sold under the name Quadramer by the company American Cyanamid. Copolymers of acrylic acid and of C₁-C₄ alkyl methacrylate and terpolymers of vinylpyrrolidone, of acrylic acid and of methacrylate of C₁-C₂₀ alkyl, for example of lauryl, such as the product sold by the company ISP under the name Acrylidone® LM and methacrylic acid/ethyl acrylate/tert-butyl acrylate terpolymers such as the product sold under the name Luvimer® 100 P by the company BASF may also be used. Methacrylic acid/acrylic acid/ethyl acrylate/methyl methacrylate copolymers as an aqueous dispersion, sold under the name Amerhold® DR 25 by the company Amerchol may also be used.

(C) crotonic acid copolymers may be used, such as those comprising vinyl acetate or propionate units in their chain and optionally other monomers such as allylic esters or methallylic esters, vinyl ether or vinyl ester of a linear or branched saturated carboxylic acid with a long hydrocarbon chain such as those comprising at least 5 carbon atoms. These polymers are optionally grafted or crosslinked. Other vinyl, allylic and methallylic ester monomers of an α- or β-cyclic carboxylic acid may also be used. Such polymers are described, for example, in French Patent Nos. 1,222,944; 1,580,545; 2,265,782, 2,265,781; 1,564,110; and 2,439,798. Commercial products falling into this class include the resins 28-29-30, 26-13-14 and 28-13-10 sold by the company National Starch.

(D) copolymers derived from C₄-C₈ monounsaturated carboxylic acids and anhydrides chosen from:
copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and acrylic acid esters, the anhydride functions of these copolymers optionally being monoesterified or monoamidated. Such polymers are described in U.S. Pat. Nos. 2,047,398, 2,723,248 and 2,102,113 and G.B. Patent No. 839,805. Commercial products that may be used include those sold under the names Gantreze AN and ES by the company ISP,
copolymers comprising (i) one or more maleic, citraconic or itaconic anhydride units and (ii) one or more monomers chosen from allylic or methallylic esters optionally comprising one or more acrylamide, methacrylamide, α-olefin, acrylic or methacrylic ester, acrylic or methacrylic acid, or vinylpyrrolidone groups in their chain, the anhydride functions of these copolymers optionally being monoesterified or monoamidated. These polymers are described, for example, in French Patent Nos. 2,350,384 and 2,357,241, and

(E) Polyacrylamides Comprising Carboxylate Groups.

Homopolymers and copolymers comprising sulfonic groups may be polymers comprising vinylsulfonic, styrenesulfonic, naphthalenesulfonic or acrylamidoalkylsulfonic units.

These polymers may be chosen from:
polyvinylsulfonic acid salts having a molecular mass ranging from approximately 1000 to 100,000, as well as copolymers having an unsaturated comonomer such as acrylic or methacrylic acids and esters thereof, as well as acrylamide or its derivatives, vinyl ethers and vinylpyrrolidone;
polystyrenesulfonic acid salts such as the sodium salts that are sold for example under the names Flexan® 500 and Flexan® 130 by National Starch. These compounds are described in French Patent No. 2,198,719;
polyacrylamidesulfonic acid salts, such as those disclosed in U.S. Pat. No. 4,128,631 and polyacrylamidoethylpropanesulfonic acid sold under the name Cosmedia Polymer HSP 1180 by Henkel.

Another anionic fixing polymer that may be used is the branched block anionic polymer sold under the name Fixate G-100 by the company Noveon.

The anionic fixing polymers may be chosen from acrylic acid copolymers, such as the acrylic acid/ethyl acrylate/N-tert-butylacrylamide terpolymers sold under the name Ultrahold® Strong by the company BASF; copolymers derived from crotonic acid, such as vinyl acetate/vinyl tert-butylbenzoate/crotonic acid terpolymers and crotonic acid/vinyl acetate/vinyl neododecanoate terpolymers sold under the name Resin 28-29-30 by the company National Starch; polymers derived from maleic, fumaric or itaconic acids or anhydrides with vinyl esters, vinyl ethers, vinyl halides, phenylvinyl derivatives and acrylic acid and esters thereof, such as methyl vinyl ether/monoesterified maleic anhydride copolymers sold, for example, under the name Gantrez® ES 425 by the company ISP; copolymers of methacrylic acid and of methyl methacrylate sold under the name Eudragit® L by the company Rohm Pharma; copolymers of methacrylic acid and of ethyl acrylate sold under the name Luvimer® MAEX or MAE by the company BASF; vinyl acetate/crotonic acid copolymers sold under the name Luviset CA 66 by the company BASF; vinyl acetate/crotonic acid copolymers grafted with polyethylene glycol sold under the name Aristoflex® A by the company BASF; and the polymer sold under the name Fixate G-100 by the company Noveon.

In some embodiments, the anionic fixing polymers used are chosen from methyl vinyl ether/monoesterified maleic anhydride copolymers sold under the name Gantrez® ES 425 by the company ISP, acrylic acid/ethyl acrylate/N-tert-butylacrylamide terpolymers sold under the name Ultrahold® Strong by the company BASF, copolymers of methacrylic acid and of methyl methacrylate sold under the name Eudragit L® by the company Rohm Pharma, vinyl acetate/vinyl tert-butylbenzoate/crotonic acid terpolymers and the crotonic acid/vinyl acetate/vinyl neododecanoate terpolymers sold under the name Resin 28-29-30 by the company National Starch, copolymers of methacrylic acid and of ethyl acrylate sold under the name Luvimer® MAEX or MAE by the company BASF, vinylpyrrolidone/acrylic acid/lauryl methacrylate terpolymers sold under the name Acrylidone® LM by the company ISP, and the polymer sold under the name Fixate G-100 by the company Noveon.

### Cationic Fixing Polymers

Cationic fixing film-forming polymers may be chosen from polymers comprising primary, secondary, tertiary and/or quaternary amine groups forming part of the polymer chain or directly attached thereto, and having a molecular weight ranging from 500 to 5,000,000, for example, from 1000 to 3,000,000.

The cationic fixing polymers may be chosen from homopolymers or copolymers of acrylic or methacrylic esters or amides comprising amine functions, cationic polysaccharides, quaternary copolymers of vinylpyrrolidone and of vinylimidazole, and chitosans.

These polymers include the following cationic polymers:
(1) homopolymers and copolymers derived from acrylic or methacrylic esters or amides and comprising at least one of the units of the following formulae: 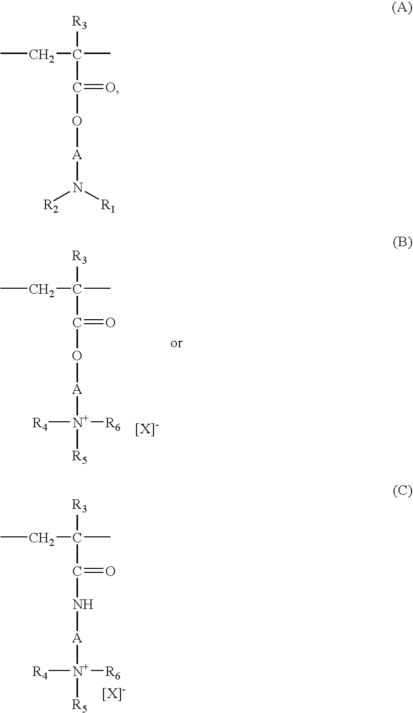
wherein:
R₃ is chosen from a hydrogen atom and a CH₃ radical;
A is chosen from a linear and branched alkyl groups comprising from 1 to 6 carbon atoms and hydroxyalkyl groups comprising from 1 to 4 carbon atoms;
R₁ and R₂, which may be identical or different, each are chosen from a hydrogen atom and alkyl groups having from 1 to 6 carbon atoms;
R₄, R₅ and R₆, which may be identical or different, each are chosen from alkyl groups having from 1 to 18 carbon atoms and a benzyl radical; and
X is chosen from a methosulfate anion and a halide such as chloride or bromide.

The copolymers of family (1) above also comprise one or more comonomer units that may be chosen from the family of acrylamides, methacrylamides, diacetone acrylamides, acrylamides and methacrylamides substituted on the nitrogen with lower (C₁₋₆) alkyl groups, groups derived from acrylic or methacrylic acids or esters thereof, vinyllactams such as vinylpyrrolidone or vinylcaprolactam, and vinyl esters.

Copolymers of family (1) above include:
copolymers of acrylamide and of dimethylaminoethyl methacrylate quaternized with dimethyl sulfate or with a dimethyl halide, such as the product sold under the name Hercofloc® by the company Hercules,
copolymers of acrylamide and of methacryloyloxyethyltrimethylammonium chloride, described, for example, in EP Patent Application No. 80,976 A and sold under the name Bina Quat P 100 by the company Ciba Geigy,
copolymers of acrylamide and of methacryloyloxyethyltrimethylammonium methosulfate, such as the product sold under the name Reten by the company Hercules,
quaternized or non-quaternized vinylpyrrolidone/dialkylaminoalkyl acrylate or methacrylate copolymers, such as the products sold under the name Gafquat® by the company ISP, such as, for example, Gafquat® 734 or Gafquat® 755, or alternatively the products known as Copolymer® 845, 958 and 937. These polymers are described in detail in French Patent Nos. 2,077,143 and 2,393,573,
fatty-chain polymers comprising a vinylpyrrolidone unit, such as the products sold under the name Styleze W20 and Styleze W10 by the company ISP,
dimethylaminoethyl methacrylate/vinylcaprolactam/vinylpyrrolidone terpolymers, such as the product sold under the name Gaffix VC 713 by the company ISP, and
quaternized vinylpyrrolidone/dimethylaminopropylmethacrylamide copolymers, such as the products sold under the name Gafquat® HS 100 by the company ISP;
(2) cationic polysaccharides, for example, comprising quaternary ammonium, such as those described in U.S. Pat. Nos. 3,589,578 and 4,031,307, such as guar gums comprising trialkylammonium cationic groups. Such products are sold under the trade names Jaguar C13 S, Jaguar C 15, and Jaguar C 17 by the company Meyhall;
(3) quaternary copolymers of vinylpyrrolidone and of vinylimidazole;
(4) chitosans and salts thereof; the salts that may be used are, for example, chitosan acetate, lactate, glutamate, gluconate or pyrrolidonecarboxylate.

These compounds include chitosan having a degree of deacetylation of 90.5% by weight, sold under the name Kytan Brut Standard by the company Aber Technologies, and chitosan pyrrolidonecarboxylate sold under the name Kytamer® PC by the company Amerchol.

(5) cationic cellulose derivatives such as copolymers of cellulose and of cellulose derivatives grafted with a water-soluble monomer comprising a quaternary ammonium, and disclosed in U.S. Pat. No. 4,131,576, such as hydroxyalkylcelluloses, for example, hydroxymethyl-, hydroxyethyl- and hydroxypropylcelluloses grafted in particular with a methacryloyloxyethyltrimethylammonium, methacrylamidopropyltrimethylammonium or dimethyldiallylammonium salt.

The products sold corresponding to this definition include the products sold under the name Celquat L 200 and Celquat H 100 by the company National Starch.

The amphoteric fixing polymers may be chosen from polymers comprising units B and C distributed randomly in the polymer chain, wherein B is a unit derived from a monomer comprising at least one basic nitrogen atom and C is a unit derived from an acid monomer comprising one or more carboxylic or sulfonic groups, or alternatively B and C can denote groups derived from carboxybetaine or sulfobetaine zwitterionic monomers;
B and C may also be a cationic polymer chain comprising primary, secondary, tertiary or quaternary amine groups, wherein at least one of the amine groups bears a carboxylic or sulfonic group connected via a hydrocarbon group or alternatively B and C form part of a chain of a polymer comprising an α,β-dicarboxylic ethylene unit wherein one of the carboxylic groups reacted with a polyamine comprising one or more primary or secondary amine groups.

### Amphoteric Fixing Polymers

The amphoteric fixing polymers may be chosen from copolymers comprising acidic vinyl units and basic vinyl units, crosslinked and acylated polyamino amides, polymers comprising zwitterionic units, chitosan-based polymers, modified (C₁-C₅)alkyl vinyl ether/maleic anhydride copolymers, amphoteric polyurethanes and amphoteric grafted silicone polymers.

Examples of amphoteric fixing polymers corresponding to the definition given above include:

(1) copolymers having acidic vinyl and basic vinyl units, such as those resulting from the copolymerization of a monomer derived from a vinyl compound bearing a carboxylic group such as acrylic acid, methacrylic acid, maleic acid, and α-chloroacrylic acid, and a basic monomer derived from a substituted vinyl compound comprising at least one basic atom, such as, dialkylaminoalkyl methacrylate and acrylate, dialkylaminoalkylmethacrylamides and -acrylamides. Such compounds are described in U.S. Pat. No. 3,836,537.

(2) polymers comprising units derived from:
(a) at least one monomer chosen from acrylamides and methacrylamides substituted on the nitrogen atom with an alkyl group,
(b) at least one acidic comonomer comprising one or more reactive carboxylic groups, and
(c) at least one basic comonomer such as esters comprising primary, secondary, tertiary and quaternary amine substituents of acrylic and methacrylic acids and the product of quaternization of dimethylaminoethyl methacrylate with dimethyl or diethyl sulfate.

N-substituted acrylamides and methacrylamides that may be used include compounds wherein the alkyl groups contain from 2 to 12 carbon atoms such as N-ethylacrylamide, N-tert-butylacrylamide, N-tert-octylacrylamide, N-octylacrylamide, N-decylacrylamide, N-dodecylacrylamide and the corresponding methacrylamides.

Acidic comonomers include acrylic acid, methacrylic acid, crotonic acid, itaconic acid, maleic acid and fumaric acid and alkyl monoesters having from 1 to 4 carbon atoms, of maleic or fumaric acids or anhydrides.

Basic comonomers include aminoethyl, butylaminoethyl, N,N′-dimethylaminoethyl and N-tert-butylaminoethyl methacrylates.

Other copolymers that may be used include octylacrylamide/acrylates/butylaminoethyl methacrylate copolymer (CTFA, 4th edition, 1991, name), such as the products sold under the names Amphomer® and Lovocryl® 47 by the company National Starch.

(3) crosslinked and acylated polyamino amides partially or totally derived from polyamino amides of the general formula: 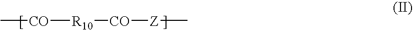
wherein:
R₁₀ is chosen from a divalent group derived from a saturated dicarboxylic acid, a mono- or dicarboxylic aliphatic acid comprising an ethylenic double bond, an ester of a lower alkanol, having from 1 to 6 carbon atoms; and a group derived from the addition of any one of said acids to a bis(primary) or bis(secondary) amine; and
Z is chosen from a group derived from a bis(primary), mono- or bis(secondary) polyalkylene-polyamine and in some embodiments, represents:
   (a) in proportions ranging from 60 to 100 mol %, the group: 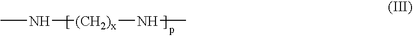
wherein x=2 and p=2 or 3, or alternatively, x=3 and p=2,
this group being derived from diethylenetriamine, from triethylenetetraamine or from dipropylenetriamine;
   (b) in proportions ranging from 0 to 40 mol %, the group (III) above wherein x=2 and p=1 and which is derived from ethylenediamine, or the group derived from piperazine: 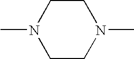
   (c) in proportions ranging from 0 to 20 mol %, the —NH(CH₂)₆—NH— group being derived from hexamethylenediamine,
these polyamino amides being crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyamino amide and acylated by the action of acrylic acid, chloroacetic acid or an alkane sultone, and salts thereof.

The saturated carboxylic acids may be chosen from acids having from 6 to 10 carbon atoms, such as adipic acid, 2,2,4-trimethyladipic acid, and 2,4,4-trimethyladipic acid, terephthalic acid, acids comprising an ethylenic double bond such as acrylic acid, methacrylic acid and itaconic acid.

The alkane sultones used in the acylation may be propane sultone or butane sultone; the salts of the acylating agents may be the sodium or potassium salts.

(4) polymers comprising zwitterionic units of the formula: 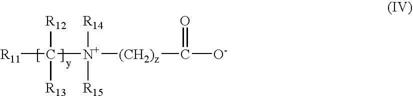
wherein:
R₁₁ is a polymerizable unsaturated group such as an acrylate, methacrylate, acrylamide or methacrylamide group,
y and z are each independently an integer ranging from 1 to 3,
R₁₂ and R₁₃ each independently are chosen from a hydrogen atom, and a methyl, ethyl and propyl group,
R₁₄ and R₁₅ each independently are chosen from a hydrogen atom and an alkyl group such that the sum of the carbon atoms in R₁₄ and R₁₅ does not exceed 10.

The polymers comprising such units may also comprise units derived from non-zwitterionic monomers such as dimethyl- or diethylaminoethyl acrylate, methacrylate or alkyl acrylates, methacrylates, acrylamides, methacrylamides, and vinyl acetate.

Examples include copolymers of methyl methacrylate/methyl dimethylcarboxymethylammonioethyl methacrylate such as the product sold under the name Diaformer Z301 by the company Sandoz.

(5) polymers derived from chitosan comprising monomer units having the following formulae: 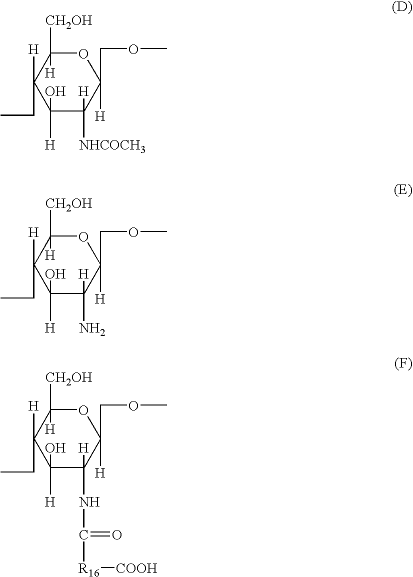
the unit (D) being present in a proportion ranging from 0 to 30%, the unit (E) in a proportion ranging from 5 to 50%, and the unit (F) in a proportion ranging from 30 to 90%, so long as, in this unit (F), R₁₆ represents a group of the formula: 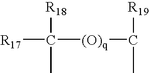
wherein, if q=0; R₁₇, R₁₈ and R₁₉, which may be identical or different, each are chosen from a hydrogen atom; a methyl, hydroxyl, acetoxy and amino residues; a monoalkylamine residue or a dialkylamine residue that is optionally interrupted by one or more nitrogen atoms and/or optionally substituted with one or more amine, hydroxyl, carboxyl, alkylthio and sulfonic groups; and an alkylthio residue wherein the alkyl group bears an amino residue, at least one of the groups R₁₇, R₁₈ and R₁₉ being, in this case, a hydrogen atom;
or, if q=1; R₁₇, R₁₈ and R₁₉ each represent a hydrogen atom,
   as well as the salts formed by these compounds with bases or acids.

(6) polymers corresponding to the general formula (V) that are described, for example, in French Patent No. 1,400,366: 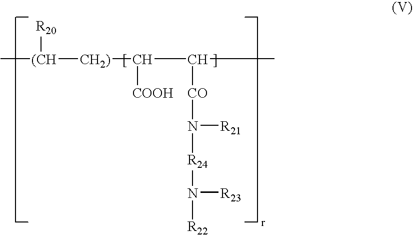
wherein:
r is an integer greater than 1,
R₂₀ is chosen from a hydrogen atom and a CH₃O, CH₃CH₂O and phenyl group,
R₂₁ is chosen from a hydrogen atom and a lower alkyl group such as methyl and ethyl,
R₂₂ is chosen from a hydrogen atom and a C₁₆ lower alkyl group such as methyl and ethyl,
R₂₃ is chosen from a C₁₋₆ lower alkyl group such as methyl and ethyl and a group corresponding to the formula: —R₂₄—N(R₂₂)₂, wherein R₂₄ is chosen from a —CH₂—CH₂—, —CH₂—CH₂—CH₂—, and a —CH₂—CH(CH₃)— group, and R₂₂ is as described above,
R₂₄ is chosen from a —CH₂—CH₂—, —CH₂—CH₂—CH₂—, and a —CH₂—CH(CH₃)— group.

(7) polymers derived from the N-carboxyalkylation of chitosan, such as N-carboxymethylchitosan and N-carboxybutylchitosan sold under the name Evalsan by the company Jan Dekker.

(8) amphoteric polymers of the type -D-X-D-X— chosen from:
(a) polymers obtained by the action of chloroacetic acid or sodium chloroacetate on compounds comprising at least one unit of formula:
   -D-X-D-X-D- (VI)
   wherein:
D is the group: 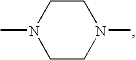
X is the symbol E or E′,
E or E′, which may be identical or different, each are chosen from a divalent group that is an alkylene group with a straight or branched chain having up to 7 carbon atoms in the main chain, which is unsubstituted or substituted with a hydroxyl group or groups and which may comprise, in addition to oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine, alkenylamine, hydroxyl, benzylamine, amine oxide, quaternary ammonium, amide, imide, alcohol, ester, and/or urethane groups;
(b) polymers of the formula:
   -D-X-D-X— (VI′)
   wherein:
D is a group of the formula: 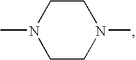
X is E or E′, and
wherein there is at least once E′;
E has the meaning given above, and
E′ is a divalent group that is an alkylene group with a straight or branched chain having up to 7 carbon atoms in the main chain, which is unsubstituted or substituted with one or more hydroxyl groups and comprising one or more nitrogen atoms, the nitrogen atom being substituted with an alkyl chain that is optionally interrupted by an oxygen atom and comprising one or more carboxyl functions or one or more hydroxyl functions and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers may also comprise other vinyl comonomers such as vinylcaprolactam.

Amphoteric fixing polymers that may be used may include those of family (3), such as the copolymers whose CTFA name is octylacrylamide/acrylates/butylaminoethyl methacrylate copolymer, such as the products sold under the names Amphomer®, Amphomer® LV 71 and Lovocryl® 47 by the company National Starch and those of family (4) such as the copolymers of methyl methacrylate/methyl dimethylcarboxymethylammonioethyl methacrylate, sold, for example, under the name Diaformer Z301 by the company Sandoz.

### Nonionic Fixing Polymers

Nonionic fixing polymers that may be used may be chosen, for example, from:
polyalkyloxazolines;
vinyl acetate homopolymers;
vinyl acetate copolymers, for example, copolymers of vinyl acetate and of acrylic ester; copolymers of vinyl acetate and of ethylene, or copolymers of vinyl acetate and of maleic ester, for example of dibutyl maleate;
homopolymers and copolymers of acrylic esters, for example, copolymers of alkyl acrylates and of alkyl methacrylates, such as the products sold by the company Rohm & Haas under the names Primal® AC-261 K and Eudragit® NE 30 D, by the company BASF under the name 8845, or by the company Hoechst under the name Appretan® N9212;
copolymers of acrylonitrile and of a nonionic monomer chosen, for example, from butadiene and alkyl (meth)acrylates; mention may be made of the products sold under the name CJ 0601 B by the company Rohm & Haas;
styrene homopolymers;
styrene copolymers, for instance copolymers of styrene and of an alkyl (meth)acrylate, such as the products Mowilith® LDM 6911, Mowilith® DM 611 and Mowilith® LDM 6070 sold by the company Hoechst, and the products Rhodopase SD 215 and Rhodopas® DS 910 sold by the company Rhône-Poulenc; copolymers of styrene, of alkyl methacrylate and of alkyl acrylate; copolymers of styrene and of butadiene; or copolymers of styrene, of butadiene and of vinylpyridine;
polyamides;
vinyllactam homopolymers other than vinylpyrrolidone homopolymers, such as the polyvinylcaprolactam sold under the name Luviskol® Plus by the company BASF; and
vinyllactam copolymers such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVPVA® S630L by the company ISP, Luviskol® VA 73, VA 64, VA 55, VA 37 and VA 28 by the company BASF; and poly(vinylpyrrolidone/vinyl acetate/vinyl propionate) terpolymers, for example, the product sold under the name Luviskol® VAP 343 by the company BASF.

In some embodiments, the alkyl groups of the nonionic polymers mentioned above may contain from 1 to 6 carbon atoms.

### Grafted Silicone Fixing Polymers

It is also possible to use grafted silicone fixing polymers comprising a polysiloxane portion and a portion comprising a non-silicone organic chain, one of the two portions constituting the main chain of the polymer, and the other being grafted onto the said main chain. These polymers are described, for example, in EP Patent Application Nos. 412,704 A, 412,707 A, 640,105 A, 582,152 A; PCT Publication Nos. WO 95/00578, and WO 93/23009, and U.S. Pat. Nos. 4,693,935, 4,728,571 and 4,972,037.

These polymers may be amphoteric, anionic or nonionic, for example, anionic or nonionic.

Such polymers include copolymers that can be obtained by free radical polymerization from the monomer mixture formed from:
(a) 50 to 90% by weight of tert-butyl acrylate;
(b) 0 to 40% by weight of acrylic acid;
(c) 5 to 40% by weight of a silicone macromer of formula: 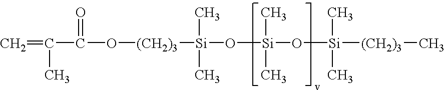
   wherein v is a number ranging from 5 to 700, the weight percentages being calculated relative to the total weight of the monomers.

Other examples of grafted silicone polymers include polydimethylsiloxanes (PDMSs) onto which are grafted, via a thiopropylene-type connecting chain, mixed polymer poly(meth)acrylic acid units and polyalkyl (meth)acrylate units and polydimethylsiloxanes (PDMSs) onto which are grafted, via a thiopropylene connecting chain, polyisobutyl (meth)acrylate polymer units.

Another type of silicone fixing polymer that may be used is the product Luviflex® Silk, sold by the company BASF.

### Polyurethane Fixing Polymers

Functionalized or non-functionalized, silicone or non-silicone, cationic, nonionic, anionic or amphoteric polyurethanes or mixtures thereof may also be used as fixing polymers.

Suitable polyurethanes include those disclosed in EP Patent Application Nos. 751,162, 637,600, and 648,485 and FR Patent Application No. 2,743,297, assigned to L'Oréal S.A., Patent Application Nos. 656,021 and WO 94/03510 from the company BASF and EP Patent Application No. 619,111 from the company National Starch.

Polyurethanes that may be used include the products sold under the names Luviset Pur® and Luviset® Si Pur by the company BASF.

The concentration of additional fixing polymer used in the compositions may range from 0.1 to 20%, for example, from 0.5 to 10% by weight relative to the total weight of the composition.

Additional Thickening Polymers

The compositions may also comprise as an additional cosmetic adjuvant at least one additional thickening polymer, also known as a “rheology modifier,” other than the polyurethanes.

The rheology modifiers may be chosen from fatty acid amides (e.g., coconut monoethanolamide or diethanolamide, or oxyethylenated carboxylic acid alkyl ether monoethanolamide), cellulose-based thickeners (e.g., hydroxyethylcellulose, hydroxypropylcellulose or carboxymethylcellulose), guar gums and derivatives thereof, such as hydroxypropyl guar gum, gums of microbial origin (e.g., xanthan gum or scleroglucan gum), acrylic acid or acrylamidopropanesulfonic acid crosslinked homopolymers, and associative polymers.

The associative polymers may be chosen from water-soluble polymers that are capable, in an aqueous medium, of reversibly associating with each other or with other molecules.

The chemical structure of the associative polymers comprises hydrophilic zones and hydrophobic zones having at least one fatty chain.

The associative polymers that may be used may be anionic, cationic, amphoteric or nonionic.

The concentration of additional thickening polymer may range from 0.01 to 20%, for example, from 0.05 to 10%, by weight relative to the total weight of the composition.

Additional Silicone and Non-Silicone Adjuvants

The compositions may also comprise as an additional cosmetic adjuvant at least one compound chosen from silicones, silicone fatty substances, and non-silicone fatty substances.

The silicones may be linear, cyclic, branched or unbranched, and volatile or non-volatile. They may be in soluble, dispersed or microdispersed form and may be in the form of oils, resins or gums, for example, polyorganosiloxanes that are insoluble in the cosmetically acceptable medium.

The organopolysiloxanes are defined in greater detail in Walter Noll's “Chemistry and Technology of Silicones” (1968) Academic Press. They may be volatile or non-volatile.

When the silicones are volatile, they may have a boiling point ranging from 60 to 260° C., and may be chosen from:
(i) cyclic silicones having from 3 to 7 (e.g., 4 to 5) silicon atoms. These are, for example, octamethylcyclotetrasiloxane sold under the name Volatile Silicone 7207 by Union Carbide or Silbione 70045 V 2 by Rhodia, decamethylcyclopentasiloxane sold under the name Volatile Silicone 7158 by Union Carbide, Silbione 70045 V 5 by Rhodia, and mixtures thereof.

Dimethylsiloxane/methylalkylsiloxane cyclocopolymers may also be used, such as Volatile Silicone FZ 3109 sold by the company Union Carbide, having the chemical structure:

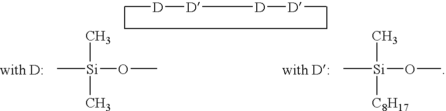

Mixtures of cyclic silicones with organosilicon compounds may be used, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;
(ii) linear volatile silicones having from 2 to 9 silicon atoms and having a viscosity of less than or equal to 5×10⁻⁶ m²/s at 25° C. An example is decamethyltetrasiloxane sold under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics.”

Non-volatile silicones that may be used include polyalkylsiloxanes, polyarylsiloxanes, polyalkylarylsiloxanes, silicone gums and resins, polyorganosiloxanes modified with organofunctional groups, and mixtures thereof.

Organomodified silicones that may be used include silicones as defined above and comprising one or more organofunctional groups attached via a hydrocarbon-based group.

Organomodified silicones that may be used include polyorganosiloxanes comprising:
polyethyleneoxy and/or polypropyleneoxy groups optionally comprising C₆-C₂₄ alkyl groups, such as the products known as dimethicone copolyol sold by the company Dow Corning under the name DC 1248 or the oils Silwet® L 722, L 7500, L 77 and L 711 from the company Union Carbide and the (C₁₂)alkylmethicone copolyol sold by the company Dow Corning under the name Q2 5200;
substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, for example, C₁-C₄ aminoalkyl groups;
thiol groups such as the products sold under the names GP 72 A and GP 71 from Genesee;
alkoxylated groups such as the product sold under the name Silicone Copolymer F-755 by SWS Silicones and Abil Wax® 2428, 2434 and 2440 by the company Goldschmidt;
hydroxylated groups such as the polyorganosiloxanes comprising a hydroxyalkyl function, described in French Patent Application No. 85/16334 A;
acyloxyalkyl groups such as the polyorganosiloxanes described in U.S. Pat. No. 4,957,732;
carboxylic acid anionic groups, such as, the products described in EP Patent No. 186,507 from the company Chisso Corporation, or of alkylcarboxylic type, such as those in the product X-22-3701 E from the company Shin-Etsu; 2-hydroxyalkyl sulfonate; 2-hydroxyalkyl thiosulfate such as the products sold by the company Goldschmidt under the names Abil® S201 and Abil® S255;
hydroxyacylamino groups, such as the polyorganosiloxanes described in EP Patent Application No. 342,834. Examples include the product Q2-8413 from the company Dow Corning.

Silicone oils that may be used in the compositions include volatile and non-volatile polymethylsiloxanes comprising a linear or cyclic silicone chain, which are liquid or pasty at room temperature, for example, cyclopolydimethylsiloxanes (cyclomethicones) such as cyclohexasiloxane; polydimethylsiloxanes comprising alkyl, alkoxy or phenyl groups, which are pendent or at the end of a silicone chain, these groups having from 2 to 24 carbon atoms; phenylsilicones, for example, phenyltrimethicones, phenyldimethicones, phenyltrimethylsiloxydiphenylsiloxanes, diphenyldimethicones, diphenylmethyldiphenyltrisiloxanes, 2-phenylethyltrimethyl siloxysilicates, polymethylphenylsiloxanes, and mixtures thereof.

Silicone gums that may be used in the compositions include polydiorganosiloxanes having a high molecular mass, i.e., ranging from 200,000 to 2,000,000, used alone or as a mixture in a solvent chosen from volatile silicones, polydimethylsiloxane oils, polyphenylmethylsiloxane oils, polydiphenyldimethylsiloxane oils, isoparaffins, methylene chloride, pentane and hydrocarbons, and mixtures thereof.

In some embodiments, a silicone gum with a molecular weight of less than 1,500,000 is used. Silicone gums include polydimethylsiloxanes, polyphenylmethylsiloxanes, poly(diphenylsiloxanedimethylsiloxanes), poly(dimethylsiloxanemethylvinylsiloxanes), poly(dimethylsiloxanephenylmethylsiloxanes), and poly(diphenylsiloxanedimethylsiloxanemethylvinylsiloxanes).

The silicone gums may be terminated at a chain end with trimethylsilyl or dimethylhydroxysilyl groups.

Silicone resins that may be used include crosslinked siloxane systems comprising (R)₂SiO_{2/2}, RSiO_{3/2} or SiO_{4/2} units, wherein R is chosen from a hydrocarbon-based group having from 1 to 6 carbon atoms and a phenyl group. In some embodiments, R is a lower (C₁-C₆) alkyl radical or a phenyl radical.

Non-silicone fatty substances that may be used in the compositions include all natural or synthetic, organic or mineral non-silicone oils, waxes, and resins.

As used herein, an oil is a lipophilic compound that is liquid at room temperature (about 25° C.), with a reversible solid/liquid change of state. Animal oils and plant oils comprise propane-1,2,3-triol triesters as essential constituents.

Oils that may be used in the compositions include:
hydrocarbon-based oils of animal origin, such as perhydrosqualene;
hydrocarbon-based oils of plant origin, such as liquid triglycerides of fatty acids having from 4 to 10 carbon atoms, e.g., heptanoic or octanoic acid triglycerides or alternatively, for example, sunflower oil, maize oil, soybean oil, marrow oil, grapeseed oil, sesame oil, hazelnut oil, apricot oil, macadamia oil, arara oil, sunflower oil, castor oil, avocado oil, caprylic/capric acid triglycerides, for example, those sold by the company Stearineries Dubois or those sold under the names Miglyol 810, 812 and 818 by the company Dynamit Nobel, jojoba oil, and shea butter oil;
synthetic esters and synthetic ethers, for example, synthetic esters and synthetic ethers of fatty acids, such as oils of formulae R₆COOR₇ and R₆OR₇ wherein R₆ is a fatty acid residue having from 8 to 29 carbon atoms and R₇ is a branched or unbranched hydrocarbon-based chain having from 3 to 30 carbon atoms, such as, purcellin oil, isononyl isononanoate, isopropyl myristate, 2-ethylhexyl palmitate, 2-octyldodecyl stearate, 2-octyidodecyl erucate or isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for example, propylene glycol dioctanoate, neopentyl glycol diheptanoate and diethylene glycol diisononanoate; and pentaerythritol esters, for example, pentaerythrityl tetraisostearate;
linear or branched hydrocarbons of mineral or synthetic origin, such as volatile or non-volatile liquid paraffins and derivatives thereof, petroleum jelly, polydecenes, and hydrogenated polyisobutene such as parleam oil;
fluid fatty alcohols having from 8 to 26 carbon atoms, for example, octyldodecanol, 2-butyloctanol, oleyl alcohol, linoleyl alcohol, and linolenyl alcohol;
alkoxylated and ethoxylated fatty alcohols such as oleth-12;
partially hydrocarbon-based fluoro oils, for example, those described in JP 2,295,912 A. Examples of fluoro oils include perfluoromethylcyclopentane and perfluoro-1,3-dimethylcyclohexane, sold under the names Flutec PC1® and Flutec PC3® by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, bromoperfluorooctyl sold under the name Foralkyl® by the company Atochem; nonafluoromethoxybutane sold under the name MSX 4518® by the company 3M and nonafluoroethoxyisobutane; perfluoromorpholine derivatives, such as the 4-trifluoromethylperfluoromorpholine sold under the name PF 5052® by the company 3M.

In the list of oils mentioned above, the term “hydrocarbon-based oil” means any oil predominantly comprising carbon and hydrogen atoms, and optionally ester, ether, fluoro, carboxylic acid and/or alcohol groups.

As used herein, “wax” means lipophilic compound that is solid at room temperature (about 25° C.), with a reversible solid/liquid change of state, having a melting point of greater than about 40° C. and which may be up to 200° C., and having an anisotropic crystal organization in the solid state. As essential constituents, the animal and plant waxes comprise carboxylic acid esters of long-chain alcohols. In general, the size of the wax crystals is such that crystals scatter and/or diffuse light, giving the composition comprising them a somewhat opaque, cloudy appearance. By raising the wax to its melting point, it is possible to make it miscible with oils and to form a microscopically homogeneous mixture, but on returning the temperature of the mixture to room temperature, a microscopically and macroscopically detectable recrystallization of the wax in the oils of the mixture is obtained (opalescence).

Waxes that may be used in the present invention include waxes of animal origin such as beeswax, spermaceti, lanolin wax and lanolin derivatives; plant waxes such as sunflower wax, rice wax, potato wax, carnauba wax, candelilla wax, ouricury wax, Japan wax, cocoa butter, cork fiber wax or sugarcane wax; mineral waxes, for example paraffin wax, petroleum jelly wax, lignite wax, microcrystalline waxes, ceresin or ozokerite; synthetic waxes such as polyethylene waxes and Fischer-Tropsch waxes, and mixtures thereof.

The concentration of compounds chosen from silicones, silicone fatty substances and non-silicone fatty substances ranges from 0.01 to 20%, such as from 0.05 to 10%, by weight relative to the total weight of the composition.

The styling compositions may further comprise at least one additive chosen from nonionic, anionic, cationic and amphoteric surfactants, nonionic, anionic, cationic and amphoteric additional polymers other than the fixing polymers used in the compositions according to the invention, ceramides and pseudoceramides, vitamins and provitamins, including panthenol, silicone or non-silicone water-soluble and liposoluble sunscreens, fillers and solid particles, for example, colored or uncolored mineral and organic pigments, nacreous agents and opacifiers, flakes, active particles, dyes, sequestering agents, plasticizers, solubilizers, acidifying agents, basifying agents, neutralizers, mineral and organic thickeners, antioxidants, hydroxy acids, penetrants, fragrances and preserving agents.

A person skilled in the art will be able to select optional additives and the amount thereof such that they do not harm the properties of the compositions according to the present invention.

The additives may be present in the compositions in an amount ranging from 0 to 20% by weight relative to the total weight of the composition.

The invention is illustrated in greater detail by the example described below. Other than in the example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLE

Formulation A below in accordance with the invention was prepared:

| Formulation A | % of active material |
|---|---|
| Diglycol/CHDM/isophthalates/SIP copolymer | 20 |
| (Eastman AQ 55 S - Eastman) | |
| Steareth-100/PEG-136/HMDI copolymer | 2 |
| (Nuvis FX 1100 - Sasol) | |
| Carbomer (Carbopol Ultrez 10 - Noveon) | 0.25 |
| Deionized water | qs 100 |
| Fragrance and ingredients | qs |

Eastman AQ 55 S sold by Eastman is a copolymer of diethylene glycol/1,4-cyclohexanedimethanol/isophthalate/sulfoisophthalate, and is a water-dispersible linear sulfonic polyester.

Steareth-100/PEG-136/HMDI copolymer (Nuvis FX 1100-Sasol) is a thickening polyurethane.

Operating Protocol:

2 g of the test formulation was applied to a lock of 2.7 g of natural hair 27 cm long.
The treated lock was rolled up on a curler 1 cm in diameter, to give it a shape.
The product was left to dry in the open air and then the lock was removed delicately from the curler.
The lock thus shaped was then immersed in a bath of 8 liters of salt water (3% NaCl) at room temperature, with magnetic stirring at 100 rpm.
The length of the lock was measured over time in order to evaluate the shape hold.
   Measurement of the Shape Hold:
% shape hold: (Lᵢ−L)/(Lᵢ−L₀)*100
L: Length of the curled lock at time t
L₀: Length of the curled lock after shaping and removal from the curler

Lᵢ: Length of the lock before shaping on the curler

| | |
|---|---|
| Immersion time | % shape hold |

| | | |
|---|---|---|
| 0 | | 100 |
| 35 | seconds | 100 |
| 40 | seconds | 100 |
| 10 | minutes | 100 |

What is claimed is:

1. A cosmetic composition comprising, in a cosmetically acceptable medium, at least one water-dispersible linear sulfonic polyester and at least one thickening polyurethane wherein the weight ratio of the at least one linear sulfonic polyester to the at least one thickening polyurethane greater than or equal to 2.

2. The cosmetic composition according to claim 1, wherein the water-dispersible linear sulfonic polyester is a polycondensate of at least one dicarboxylic acid or an ester thereof, at least one diol, and at least one difunctional sulfoaryldicarboxylic compound substituted on the aromatic nucleus with an —SO₃M group wherein M is chosen from a hydrogen atom and a metal ion.

3. The cosmetic composition according to claim 2, wherein M is chosen from Na⁺, Li⁺, and K⁺.

4. The cosmetic composition according to claim 1, wherein the water-dispersible linear sulfonic polyester comprises one or more units derived from isophthalic acid, sulfoaryidicarboxylic acid salt, the sodium salt of sulfoisophthalic acid, diethylene glycol and 1,4-cyclohexanedimethanol.

5. The cosmetic composition according to claim 1, wherein the Tg of the at least one water-dispersible linear sulfonic polyester is greater than or equal to 50° C.

6. The cosmetic composition according to claim 1, wherein the concentration of the at least one water-dispersible linear sulfonic polyester ranges from 0.1 to 40% by weight relative to the total weight of the composition.

7. The cosmetic composition according to claim 6, wherein the concentration of the at least one water-dispersible linear sulfonic polyester ranges from 1 to 30% by weight relative to the total weight of the composition.

8. The cosmetic composition according to claim 7, wherein the concentration of the at least one water-dispersible linear sulfonic polyester ranges from 5 to 25% by weight relative to the total weight of the composition.

9. The cosmetic composition according to claim 1, wherein the at least one thickening polyurethane is anionic, cationic, amphoteric or nonionic.

10. The cosmetic composition according to claim 1, wherein the thickening polyurethane comprises at least one fatty chain comprising at least 8 carbon atoms.

11. The cosmetic composition according to claim 1, wherein the thickening polyurethane is a cationic thickening polyurethane of formula (I):
R—X—(P)ₙ-[L-(Y)ₘ]ᵣ-L′-(P′)ₚ—X′—R′ (I)
wherein:
R and R′, which may be identical or different, are each chosen from a hydrophobic group and a hydrogen atom;
X and X′, which may be identical or different, are each chosen from a group comprising an amine function optionally bearing a hydrophobic group, and an L″ group;
L, L′ and L″, which may be identical or different, are each chosen from a group derived from a diisocyanate;
P and P′, which may be identical or different, are each chosen from a group comprising an amine function optionally bearing a hydrophobic group;
Y is a hydrophilic group;
r is an integer ranging from 1 to 100;
n, m and p each range from 0 to 1000; and
wherein the cationic thickening polyurethane comprises at least one protonated or quaternized amine function and at least one hydrophobic group.

12. The cosmetic composition according to claim 11, wherein r is an integer ranging from 1 to 50.

13. The cosmetic composition according to claim 12, wherein r is an integer ranging from 1 to 25.

14. The cosmetic composition according to claim 1, wherein the thickening polyurethane is an amphoteric thickening polyurethane chosen from polyether polyurethanes comprising hydrophilic blocks and hydrophobic blocks.

15. The cosmetic composition according to claim 14, wherein the hydrophilic blocks are polyoxyethylene blocks.

16. The cosmetic composition according to claim 15, wherein the hydrophobic blocks are chosen from aliphatic sequences, cycloaliphatic sequences, aromatic sequences, and mixtures thereof.

17. The cosmetic composition according to claim 1, wherein the at least thickening polyurethane is a nonionic thickening polyurethane.

18. The cosmetic composition according to claim 1, wherein the concentration of the at least one thickening polyurethane ranges from 0.01 to 10% by weight relative to the total weight of the composition.

19. The cosmetic composition according to claim 18, wherein the concentration of the at least one thickening polyurethane ranges from 0.05 to 5% by weight relative to the total weight of the composition.

20. The cosmetic composition according to claim 19, wherein the concentration of the at least one thickening polyurethane ranges from 0.1 to 2% by weight relative to the total weight of the composition.

21. The cosmetic composition according to claim 1, wherein the ratio of the at least one water-dispersible linear sulfonic polyester to the at least one thickening polyurethane ranges from 2 to 50%.

22. The cosmetic composition according to claim 21, wherein the ratio of the at least one water-dispersible linear sulfonic polyester to the at least one thickening polyurethane ranges from 2.5 to 20%.

23. The cosmetic composition according to claim 22, wherein the ratio of the at least one water-dispersible linear sulfonic polyester to the at least one thickening polyurethane ranges from 3 to 15%.

24. The cosmetic composition according claim 1, further comprising at least one additional fixing polymer chosen from anionic, cationic, amphoteric and nonionic fixing polymers, and mixtures thereof.

25. The cosmetic composition according to claim 24, wherein the cationic fixing polymer is chosen from homopolymers and copolymers of acrylic and methacrylic esters and amides comprising amine functions, cationic polysaccharides, quaternary copolymers of vinylpyrrolidone, quaternary copolymers of vinylimidazole, and chitosans.

26. The cosmetic composition according to claim 24, wherein the anionic fixing polymer is chosen from homopolymers and copolymers of acrylic and methacrylic acid and salts thereof, crotonic acid copolymers, copolymers of C₄-C₈ monounsaturated carboxylic acids and anhydrides, polyacrylamides comprising carboxylate groups, homopolymers and copolymers comprising sulfonic groups, anionic polyurethanes, and anionic grafted silicone polymers.

27. The cosmetic composition according to claim 24, wherein the amphoteric fixing polymer is chosen from copolymers comprising acidic vinyl units and basic vinyl units, crosslinked and acylated polyamino amides, polymers comprising zwitterionic units, chitosan-based polymers, modified (C₁-C₅)alkyl vinyl ether/maleic anhydride copolymers, amphoteric polyurethanes, and amphoteric grafted silicone polymers.

28. The cosmetic composition according to claim 24, wherein the nonionic fixing polymer is chosen from polyalkyloxazolines, vinyl acetate homopolymers and copolymers, acrylic ester homopolymers and copolymers, polyamide acrylonitrile copolymers, styrene homopolymers and copolymers, vinyllactam homopolymers other than vinylpyrrolidone homopolymers, vinyllactam copolymers, and nonionic grafted silicone polymers.

29. The cosmetic composition according to claim 24, wherein that the concentration of additional fixing polymer ranges from 0.1 to 20% by weight relative to the total weight of the composition.

30. The cosmetic composition according to claim 29, wherein that the concentration of additional fixing polymer ranges from 0.5 to 10% by weight relative to the total weight of the composition.

31. The cosmetic composition according to claim 1, further comprising an additional thickening polymer other than the at least one thickening polyurethane.

32. The cosmetic composition according to claim 31, wherein the concentration of additional thickening polymer ranges from 0.01 to 20% by weight relative to the total weight of the composition.

33. The cosmetic composition according to claim 32, wherein the concentration of additional thickening polymer ranges from 0.05 to 10% by weight relative to the total weight of the composition.

34. The cosmetic composition according to claim 1, further comprising at least one compound chosen from silicones, silicone fatty substances, and non-silicone fatty substances.

35. The cosmetic composition according to claim 34, wherein the concentration of the at least one compound chosen from silicones, silicone fatty substances, and non-silicone fatty substances ranges from 0.01 to 20% by weight relative to the total weight of the composition.

36. The cosmetic composition according to claim 35, wherein the concentration of the at least one compound chosen from silicones, silicone fatty substances, and non-silicone fatty substances ranges from 0.05 to 10% by weight relative to the total weight of the composition.

37. The cosmetic composition according to claim 1, further comprising at least one additive chosen from nonionic, anionic, cationic and amphoteric surfactants, nonionic, anionic, cationic and amphoteric additional polymers, ceramides and pseudoceramides, vitamins and provitamins, silicone or non-silicone water-soluble and liposoluble sunscreens, fillers and solid particles, nacreous agents, opacifiers, flakes, active particles, dyes, sequestering agents, plasticizers, solubilizers, acidifying agents, basifying agents, neutralizers, mineral and organic thickeners, antioxidants, hydroxy acids, penetrants, fragrances and preserving agents.

38. The cosmetic composition according to claim 37, wherein the vitamins and provitamins is panthenol and the fillers and solid particles are chosen from colored and uncolored mineral and organic pigments.

39. The cosmetic composition according to claim 1, wherein the composition does not contain any C₁-C₄ alcohol.

40. The cosmetic composition according to claim 1, wherein the composition is transparent or translucent.

41. A process for shaping, holding, and/or fixing a hairstyle comprising applying to the hair a cosmetic composition comprising, in a cosmetically acceptable medium, at least one water-dispersible linear sulfonic polyester and at least one thickening polyurethane wherein the weight ratio of the at least one linear sulfonic polyester to the at least one thickening polyurethane greater than or equal to 2 in a sufficient amount to shape, hold, and/or fix the hairstyle.

42. A method of creating a water-resistant hairstyle comprising applying to hair a cosmetic composition comprising, in a cosmetically acceptable medium, at least one water-dispersible linear sulfonic polyester and at least one thickening polyurethane wherein the weight ratio of the at least one linear sulfonic polyester to the at least one thickening polyurethane greater than or equal to 2 in a sufficient amount to result in water-resistance.
